(19) United States
(12) Patent Application Publication
(10) Pub. No.: US 2021/0389309 A1
(43) Pub. Date: Dec. 16, 2021

(54) ASSESSMENT METHOD AND DIAGNOSTIC KIT FOR PREDICTING LONG-TERM PROGNOSIS OF ACUTE CORONARY SYNDROME ASSOCIATED WITH DEPRESSION

(71) Applicant: INDUSTRY FOUNDATION OF CHONNAM NATIONAL UNIVERSITY, Gwangju (KR)

(72) Inventors: Jae Min KIM, Gwangju (KR); Hee Ju KANG, Gwangju (KR)

(21) Appl. No.: 17/168,436

(22) Filed: Feb. 5, 2021

(30) Foreign Application Priority Data
Jun. 10, 2020 (KR) 10-2020-0070457

(51) Int. Cl. G01N 33/53; G01N 33/577

(52) U.S. Cl. CPC G01N 33/5308; G01N 33/577; G01N 2800/50; G01N 2800/304; G01N 2800/52; G01N 2496/00

(57) ABSTRACT

A diagnostic method according to an embodiment of the present disclosure for determining prognosis including recurrence and/or death, which commonly occurs after acute coronary syndrome. A method of determining long-term prognosis of acute coronary syndrome according to an embodiment of the present disclosure is capable of determining the risk of incidence of major adverse cardiac events including recurrence and/or death after acute coronary syndrome in acute coronary syndrome patients suffering from depression by analyzing the extent of NR3C1 methylation.

### CROSS REFERENCE TO RELATED APPLICATION

The present application claims priority based on Korean Patent Application No. 10-2020-0070457, filed on Jun. 10, 2020, the entire content of which is incorporated herein for all purposes by this reference.

### BACKGROUND

### 1. Technical Field

The present disclosure relates to a diagnostic method for determining long-term prognosis, including recurrence and death, which commonly occurs after acute coronary syndrome, and more particularly to a method of determining long-term prognosis of acute coronary syndrome, which is capable of determining long-term prognosis including the risk of incidence of major adverse cardiac events such as recurrence and/or death in patients with acute coronary syndrome diagnosed to have depression at baseline, by analyzing the extent of NR3C1 methylation, and to a diagnostic kit therefor.

### 2. Description of the Related Art

Depression is common in patients with acute coronary syndrome (ACS; including myocardial infarction and depressive angina), and when accompanied by depression, ACS is associated with a poor prognosis and may lead to high morbidity and mortality. It is considered that a common biological mechanism for acute coronary syndrome and depression may explain the association there between. Among these, dysregulation of the hypothalamic-pituitary-adrenal axis (HPA axis) is regarded as the main pathogenesis of depression and is associated with impaired repair after cardiac injury, so it may be considered as a candidate mechanism for the association between acute coronary syndrome and depression.

The effect of cortisol, which is the main endogenous glucocorticoid hormone of the HPA axis, is mainly mediated by glucocorticoid receptors (GRs), and the expression and sensitivity of glucocorticoid receptors are regulated by epigenetic changes. DNA methylation, the most widely studied among epigenetic changes, refers to the process of covalent bonding of a methyl group to the 5th carbon of the cytosine base of a CpG dinucleotide, where cytosine is followed by guanine. Hypermethylation of the glucocorticoid receptor gene (nuclear receptor subfamily 3, group C, member 1; NR3C1) has been reported to be associated with decreased GR expression not only in animal studies, but also in studies on humans suffering from depression and post-traumatic stress disorder and exhibiting suicidal behavior. In regard to acute coronary syndrome, NR3C1 hypermethylation has been reported to be associated with atherosclerosis and increased cardiovascular reactivity. However, the effect of NR3C1 methylation on prognosis of acute coronary syndrome has not been investigated, despite the clinical significance of comorbidity of depression and acute coronary syndrome.

Meanwhile, it has been suggested that treatment with antithrombotic agents and cholesterol-reducing agents (statins) has potentials to prevent the recurrence of acute coronary syndrome to some extent. Previous clinical trials with large sample size have indicated that statins contribute to decrease of recurrence in 30% of patients with acute coronary syndrome but that treatment with the above kinds of drugs cannot prevent recurrence in the remaining 70% of patients. Hence, new treatment protocols that prevent the recurrence in all patients with acute coronary syndrome are required, but there has been no treatment yet. Under these circumstances, the prevention of recurrence through accurate identification of high-risk cases and accompanying intensive management, or the prevention of sudden recurrence in outpatients is thought to be useful for improving life expectancy and prognosis.

The prognosis of recurrence of acute coronary syndrome is clinically determined using a mechanical test, such as a cardiac catheter test, including coronary angiography or left ventricular angiography, or using a biomarker. However, the former is not easy to implement in terms of time and cost, and consequently imposes a heavy burden on patients. Moreover, these methods do not provide complete information in view of predicting recurrence.

Therefore, there is a need to develop a new biomarker that is sufficiently effective as a marker capable of predicting the long-term prognosis of acute coronary syndrome.

### SUMMARY

The inventors of the present disclosure have conducted a number of studies and ascertained that an increase in NR3C1 methylation is significantly associated with the long-term prognosis, particularly the risk of major adverse cardiac events, in acute coronary syndrome patients suffering from depression, thus culminating in the present disclosure.

Accordingly, an objective of the present disclosure is to provide a method of determining long-term prognosis of acute coronary syndrome that may contribute to a decision-making process pertaining to therapeutic drugs or treatment methods, by confirming that methylation of a specific region of the NR3C1 gene may be used as a biomarker for predicting long-term prognosis of acute coronary syndrome, making it possible to predict long-term prognosis such as incidence of major adverse cardiac events including recurrence and/or death after acute coronary syndrome.

Another objective of the present disclosure is to provide a diagnostic kit for determining long-term prognosis of acute coronary syndrome, in which, the methylation status of a specific region of NR3C1 is measured using biosamples of acute coronary syndrome patients with depression at baseline, thus predicting the incidence of major adverse cardiac events including recurrence and/or death after acute coronary syndrome in patients with acute coronary syndrome 5 years after baseline, thereby enabling the preemptive prevention of recurrence and/or death after acute coronary syndrome in patients, which is clinically useful.

The objectives of the present disclosure are not limited to the foregoing, and will be able to be clearly understood through the following description and to be realized by the means described in the claims and combinations thereof.

In order to accomplish the above objectives, the present disclosure provides a method of determining long-term prognosis of acute coronary syndrome associated with depression, including: an investigation step of confirming whether a patient with acute coronary syndrome has depression at baseline; a measurement step of measuring the level or amount of a biomarker for predicting long-term prognosis of acute coronary syndrome associated with depression contained in a biological sample of the patient confirmed to have depression in the investigation step; and a decision step of determining the risk of onset of a major adverse cardiac event including recurrence or death after acute coronary syndrome based on the level or amount of the biomarker for predicting long-term prognosis of acute coronary syndrome associated with depression measured in the measurement step.

In a preferred embodiment, the biomarker for predicting long-term prognosis of acute coronary syndrome associated with depression is methylation of a CpG region contained in a nucleotide region at 5′-end positions from −3166 to −3147 of an NR3C1 gene.

In a preferred embodiment, the decision step is performed by comparing the extent of methylation of the CpG region contained in the nucleotide region at 5′-end positions from −3166 to −3147 of the NR3C1 gene measured in the measurement step with a preset reference level, and the reference level is determined depending on the extent of methylation of three CpG sites contained in the nucleotide region at 5′-end positions from −3166 to −3147 of the NR3C1 gene obtained from a patient population with acute coronary syndrome at baseline.

In a preferred embodiment, the three CpG sites are CpG1, CpG2 and CpG3, and the reference level is 21%, determined in consideration of median and mean values of the average methylation percentage of CpG1, CpG2 and CpG3.

In a preferred embodiment, in the decision step, it is determined that there is a risk of onset of a major adverse cardiac event 5 years after baseline when the average methylation value of CpG1, CpG2 and CpG3 measured is equal to or greater than the reference level.

In a preferred embodiment, when the extent of methylation of the CpG region is increased, the risk of onset of a major adverse cardiac event is increased.

In a preferred embodiment, when the average methylation value of CpG1, CpG2, and CpG3 in the CpG region is increased by 10% over the reference level, the risk of onset of a major adverse cardiac event is increased by 11%.

In a preferred embodiment, the biological sample is selected from among a tissue and a body fluid including blood.

In addition, the present disclosure provides a diagnostic kit for determining long-term prognosis of acute coronary syndrome, including: a measurement means for measuring a biomarker for predicting long-term prognosis of acute coronary syndrome associated with depression, which measures methylation of a CpG region contained in a nucleotide region at 5′-end positions from −3166 to −3147 of an NR3C1 gene in a patient with acute coronary syndrome confirmed to have depression at baseline.

In a preferred embodiment, the measurement means uses sodium bisulfate and a polymerase chain reaction (PCR) or uses a monoclonal antibody against 5-methylcytosine.

In a preferred embodiment, the CpG region includes CpG1, CpG2 and CpG3.

In a preferred embodiment, the diagnostic kit is a microarray.

According to the present disclosure, it is confirmed that methylation of a specific region of the NR3C1 gene can be used as a biomarker for predicting long-term prognosis of acute coronary syndrome, thereby providing a biomarker that can relatively accurately determine long-term prognosis of acute coronary syndrome 5 years after baseline.

In addition, the method of determining long-term prognosis of acute coronary syndrome according to the present disclosure enables the prediction and/or diagnosis of the possibility of onset of major adverse cardiac events 5 years after baseline and can thus contribute to the decision-making process pertaining to therapeutic drugs or treatment methods.

Moreover, the extent of methylation of the specific region of the NR3C1 gene using biosamples of patients with acute coronary syndrome confirmed to have depression at baseline according to the present disclosure is measured, thus predicting the possibility of incidence of major adverse cardiac events including recurrence and/or death after acute coronary syndrome in patients with acute coronary syndrome, thereby enabling the preemptive prevention of recurrence and/or death after acute coronary syndrome in patients, which is clinically useful.

The effects of the present disclosure are not limited to the foregoing, and should be understood to include all effects that can be reasonably anticipated from the following description.

### BRIEF DESCRIPTION OF THE DRAWINGS

FIG. 1A is a flowchart showing an overview of a patient recruitment process, and FIG. 1B is a schematic diagram for analyzing the extent of methylation of the NR3C1 gene and a sequence thereof; and

FIGS. 2A to 2C are graphs showing the cumulative incidence (%) of composite major adverse cardiac event (MACE) by average NR3C1 methylation and depressive disorder at baseline.

### DETAILED DESCRIPTION

The terminology used in the present disclosure is merely used to describe particular embodiments, and is not intended to limit the present disclosure. As used herein, the singular forms are intended to include the plural forms as well, unless the context clearly indicates otherwise. It will be understood that the terms “comprise”, “include”, “have”, etc. when used in this specification specify the presence of stated features, integers, steps, operations, elements, components, or combinations thereof, but do not preclude the presence or addition of one or more other features, integers, steps, operations, elements, components, or combinations thereof.

It will be further understood that, although terms such as “first”, “second”, etc. may be used herein to describe various elements, these elements are not limited by these terms. These terms are only used to distinguish one element from another element. For instance, a “first” element discussed below could be termed a “second” element without departing from the scope of the present disclosure. Similarly, a “second” element could also be termed a “first” element.

Unless otherwise defined, all terms including technical and scientific terms used herein have the same meanings as those commonly understood by one of ordinary skill in the art to which the present disclosure belongs. It will be further understood that the terms used herein should be interpreted as having meanings consistent with their meanings in the context of this specification and the relevant art, and are not to be interpreted in an idealized or overly formal sense unless expressly so defined herein.

In interpreting elements, it is to be understood that an error range is included even if there is no separate description thereof.

In the case of a description of a temporal relationship, for example, when the temporal relationship is described as “after”, “following”, “subsequently”, “before”, etc., this includes non-consecutive cases, unless “immediately” or “directly” is used.

As used herein, the term “diagnosis” means identifying the presence or characteristic of a pathological condition. With regard to the purpose of the present disclosure, “diagnosis” means determining the long-term prognosis of acute coronary syndrome based on in-vitro analysis of body fluids, that is, the possibility of incidence of major adverse cardiac events 5 years after baseline.

As used herein, the term “biomarker” means a substance that may indicate a disease state. In the context of the present disclosure regarding the diagnosis of long-term prognosis of acute coronary syndrome, the biomarker indicates the extent of methylation of a specific region of the NR3C1 gene. Among patients with acute coronary syndrome confirmed to have depression at baseline, patients having a biomarker level or amount equal to or greater than a reference level are more likely to experience major adverse cardiac events 5 years after baseline.

As used herein, the term “blood” includes whole blood, serum and plasma.

As used herein, the term “reference level” means a relative level or an absolute level determined to distinguish individuals who are at risk of onset of major adverse cardiac events 5 years after baseline among acute coronary syndrome patients with depression at baseline. The reference level may be given as a characteristic value represented by, for example, a fold difference in the case of a relative level, or, for example, a percentage (%) in the case of an absolute level. As pointed out herein, a value equal to or greater than the reference level is regarded as enabling determination of the risk of onset of major adverse cardiac events 5 years after baseline.

As used herein, the term “predicting” means finding that an individual is significantly more likely to develop a biological disease.

As used herein, the term “biological sample” includes various types of samples obtained from an individual, and may also be used in diagnosis or monitoring analysis. Biological fluid samples include blood, cerebrospinal fluid (CSF), urine, and other liquid samples of biological origin. For example, the sample may be pretreated for concentration and separation, depending on the need.

As used herein, the term “individual” is a mammal, preferably a human, and the terms “individual” and “subject” may be used interchangeably in the present disclosure.

As used herein, the term “baseline” refers to the time point at which initial medical treatment is performed after the onset of acute coronary syndrome, and preferably a time point within 2 weeks (average 7 days) of inpatient treatment after the onset of acute coronary syndrome.

Hereinafter, a detailed description will be given of the technical configuration of the present disclosure with reference to the accompanying drawings and preferred embodiments.

However, the present disclosure is not limited to the embodiments described herein, and may be embodied in other forms. Throughout the specification, the same reference numerals used to explain the present disclosure designate the same elements.

In the present disclosure, it is confirmed that methylation of a specific region of the NR3C1 gene may be used as a biomarker for predicting long-term prognosis of acute coronary syndrome, and thus the present disclosure is intended to provide a method of determining long-term prognosis of acute coronary syndrome by measuring the extent of methylation of the specific region of the NR3C1 gene in order to predict and/or diagnose the possibility of incidence of major adverse cardiac events including recurrence and/or death after acute coronary syndrome, and a diagnostic kit therefor.

As described below, the present disclosure clearly elucidates the correlation between the extent of methylation of a specific region of the NR3C1 gene in a patient with diagnosed depression at baseline at which acute coronary syndrome has been occurred and major adverse cardiac events 5 years after baseline.

Therefore, the method of determining long-term prognosis of acute coronary syndrome according to the present disclosure includes an investigation step of confirming whether a patient with acute coronary syndrome has depression at baseline, a measurement step of measuring the level or amount of a biomarker for predicting long-term prognosis of acute coronary syndrome associated with depression contained in a biological sample of the patient confirmed to have depression in the investigation step, and a decision step of determining the risk of onset of a major adverse cardiac event including recurrence or death after acute coronary syndrome based on the level or amount of the biomarker for predicting long-term prognosis of acute coronary syndrome associated with depression measured in the measurement step.

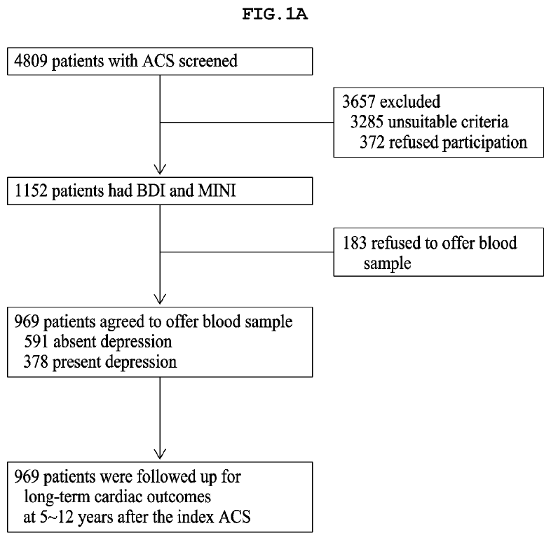

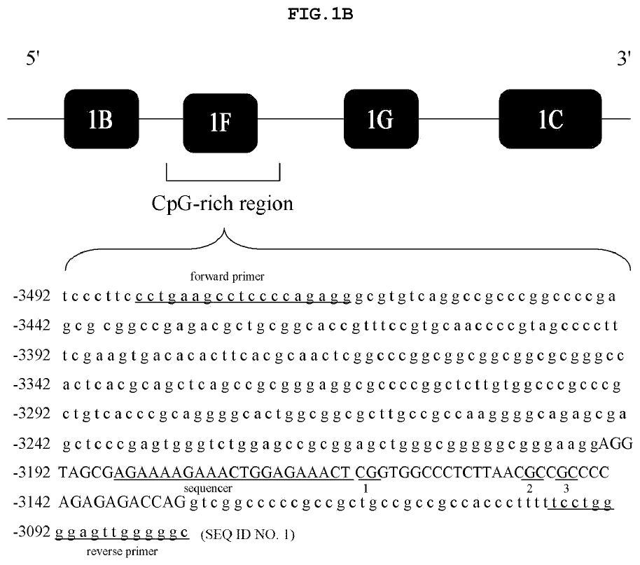

More specifically, the biomarker for predicting long-term prognosis of acute coronary syndrome associated with depression analyzed in the measurement step is the methylation of the CpG region contained in the nucleotide region at 5′-end positions from −3166 to −3147 of the NR3C1 gene, and an increase in the methylation of the CpG region may indicate the risk of onset of a major adverse cardiac event. Here, the CpG region includes CpG1, CpG2, and CpG3, and may be the nucleotide region at 5′-end positions from −3166 to −3147 in the 5′-end 1F exon region of the NR3C1 gene, as shown in FIGS. 1A and 1B. The measurement method used in the measurement step may be a known method useful for measuring the level or amount of the biomarker for predicting long-term prognosis of acute coronary syndrome associated with depression and performing determination or diagnosis based on the measured level or amount. In the present disclosure, the detection method may be performed in vitro and/or in vivo, but preferably the detection method of the present disclosure is an in-vitro method based on a sample obtained from an individual and provided in vitro.

Also, the decision step is performed by comparing the extent of methylation of the CpG region contained in the nucleotide region at 5′-end positions from −3166 to −3147 of the NR3C1 gene measured in the measurement step with a preset reference level. Here, the reference level is determined depending on the extent of methylation of three CpG sites contained in the nucleotide region at 5′-end positions from −3166 to −3147 of the NR3C1 gene obtained from a patient population suffering from acute coronary syndrome at baseline. Here, the three CpG sites are CpG1, CpG2 and CpG3, and the reference level is determined to be 21% in consideration of the median and mean values of the average methylation percentage of CpG1, CpG2 and CpG3. Therefore, in an embodiment of the present disclosure, in the decision step, it may be determined that there is a risk of onset of a major adverse cardiac event 5 years after baseline when the average methylation value of CpG1, CpG2, and CpG3 measured is equal to or greater than the reference level. Moreover, when the extent of methylation of the CpG region measured in the measurement step increases, it is predicted that the risk of onset of a major adverse cardiac event 5 years after baseline also increases. In particular, when the average methylation value of CpG1, CpG2, and CpG3 in the CpG region is increased by 10% over the reference level, it may be predicted that the risk of onset of a major adverse cardiac event is increased by 11%. In terms of the risk of a major adverse cardiac event classified by the type of prognosis, all-cause mortality was increased by 8%, cardiac death was increased by 11%, recurrent myocardial infarction was increased by 7%, and the likelihood of recurrent percutaneous coronary intervention was increased by 12%.

In addition, the present disclosure pertains to a diagnostic kit for determining long-term prognosis of acute coronary syndrome, which is used to determine the risk of onset of a major adverse cardiac event 5 years after baseline in a patient with acute coronary syndrome confirmed to have depression at baseline. The diagnostic kit of the present disclosure includes a measurement means for measuring a biomarker for predicting long-term prognosis of acute coronary syndrome associated with depression, which measures the methylation of the CpG region contained in the nucleotide region at 5′-end positions from −3166 to −3147 of the NR3C1 gene, using biological samples selected from among tissues and body fluids, including blood, of patients with acute coronary syndrome confirmed to have depression at baseline. Here, the measurement means is able to use sodium bisulfite and a polymerase chain reaction (PCR), or to use a monoclonal antibody against 5-methylcytosine. Here, it is determined that patients in which methylation of the CpG region including CpG1, CpG2, and CpG3 by the measurement means is confirmed to be equal to or greater than 21%, which is the reference level, are more likely to experience major adverse cardiac events than patients confirmed to have methylation less than the reference level. More specifically, as will be described later, the possibility of incidence of major adverse cardiac events was 1.98 times higher in patients confirmed to have methylation equal to or greater than the reference level than in patients confirmed to have methylation less than the reference level.

Among major adverse cardiac events depending on the type of prognosis, all-cause mortality was increased 1.9 times, cardiac death was increased 2.24 times, recurrent myocardial infarction was increased 1.78 times, and the likelihood of recurrent percutaneous coronary intervention was increased 1.9 times.

Also, the diagnostic kit may be implemented as a microarray.

### Examples

1. Study Subject

The analyses were performed using data from a large naturalistic cohort study named the DEPression in ACS (DEPACS) study, which was designed to investigate the correlation between depression and ACS.

For the DEPACS study entry, the inclusion criteria were as follows: i) aged 18-85 years; ii) ACS confirmed through investigation (the presence of ST-segment elevation MI was determined by >30 min of continuous chest pain, a new ST-segment elevation 2 mm in at least two contiguous electrocardiographic leads, and creatine kinase-MB more than three times normal; the presence of non-ST-segment elevation MI was diagnosed by chest pain and a positive cardiac biochemical marker without new ST-segment elevation; and the presence of unstable angina was determined by chest pain within the preceding 72 hours with or without ST-T wave changes or positive cardiac biochemical markers); iii) ability to complete study questionnaires; and iv) ability to understand the study objectives and provide informed consent. Exclusion criteria were: i) occurrence of ACS while hospitalized for another reason; ii) ACS development less than 3 months after a coronary artery bypass graft procedure; iii) uncontrolled hypertension (systolic blood pressure (BP)>180 mmHg or diastolic BP>100 mmHg); iv) resting heart rate<40/min; v) severe physical illnesses threatening life or interfering with recovery from ACS; and vi) persistent clinically significant laboratory abnormalities in complete blood cell counts, thyroid tests, renal function tests, and liver function tests.

The overview of the patient recruitment process for the present analysis is shown in FIG. 1A. 4809 ACS patients admitted to the Department of Cardiology of Chonnam National University Hospital from 2006 to 2012 were interviewed face to face and sequentially enrolled. The Department of Cardiology at Chonnam National University Hospital was authorized by the Korean Circulation Society to serve as the central coordinating center for the Korea Acute Myocardial Infarction Registry (KAMIR). KAMIR is a web-based registry platform (http://kamir5.kamir.or.kr/) to collect nationwide information from multiple centers on clinical practices and outcomes of patients with acute myocardial infarction prospectively, which enables it to evaluate the prospective associations of a range of exposure or interventions with long-term cardiac outcomes. ACS patients were treated by cardiologists participating in the study in accordance with international ACS intervention guidelines. 1152 ACS patients who met the eligibility criteria and agreed to participate in the study were received baseline evaluation during the hospitalization period within 2 weeks of onset (mean 6.3×SD 2.4 days). 969 patients, corresponding to 84.1% of the above patients, consented to the blood test and constituted the participants of this study. A follow-up evaluation in 2017 between 5 and 12 years after of the index ACS was conducted for all participants. Written consent was obtained for all participants, and this study was conducted in accordance with the 1963 Helsinki Declaration and institutional guidelines, and was approved by the Chonnam University Hospital Institutional Review Board.

2. Baseline Evaluation

Depression was diagnosed by a psychiatrist with the Mini-International Neuropsychiatric Interview (MINI), a structured diagnostic interview for psychiatric disorders based on the criteria of the Diagnostic and Statistical Manual of Mental Disorders, Fourth Edition (DSM-IV) for defining major or minor depressive disorders. In order to investigate the extent of NR3C1 methylation as described below, DNA was collected from venous blood using a standard method.

(1) NR3C1 1F Methylation

In the present disclosure, the methylation pattern of the exon 1F region of NR3C1 (GenBank #AY 436590) was investigated. In particular, three CpG sites in the exon 1F (FIG. 1B), corresponding to the first to third CpG sites described by Perroud et al. (2011) and the sixth to eighth CpG sites described by Oberlander et al. (2008), were investigated. The CpG-rich region of the NR3C1 exon 1F sequence located between −3166 and −3147 from the translation start site (1+) located 13 nucleotides from the beginning of exon 2 was analyzed, and three CpG sites contained in the above region were analyzed. These three sites were selected because their methylation status were extensively investigated to be associated with adverse life experiences and related disorders including borderline personality disorder, depression and post-traumatic stress disorder. Moreover, the relatively consistent direction of the association between methylation and early adversity was discussed in a recent review paper (Daskalakis and Yehuda, 2014).

Using blood samples obtained from participants who agreed to blood collection, the extent of DNA methylation of the NR3C1 gene was investigated as follows. Genomic DNA (1 μg) was extracted from whole blood and treated with a blood coagulant using the QIAamp DNA Blood Mini Kit (Qiagen, Valencia, Calif., USA). Subsequently, the DNA was subjected to bisulfite treatment in accordance with the manufacturer's protocol using the EpiTech Bisulfite Kit (Qiagen). The amplification of the 406-bp fragment of the NR3C1 gene was performed via polymerase chain reaction (PCR) from bisulfite-treated DNA with the forward and reverse primers shown in FIG. 1B. PCR was performed under the following conditions: 45 cycles of 94° C. for 15 min, 94° C. for 30 sec, 58° C. for 30 sec, and 72° C. for 40 sec, and a final extension of 10 min at 72° C. The PCR product was sequenced by identifying nucleotides introduced by DNA polymerase using a PSQ 96M pyrosequencing system (Biotage). Pyro Q-CpG software version 1.0.9 (Biotage) was used to quantify the methylation percentage of each CpG site. In subsequent analysis, the individual methylation percentages at the three CpG sites, namely CpG1, CpG2 and CpG3, and the average value thereof were used.

(2) Evaluation of various characteristics Various characteristics that could potentially affect cardiac outcomes were evaluated as follows. As for demographic characteristics, age, gender, education, marital status, living alone, housing (owned or rented), and employment status (employed) were evaluated. As clinical characteristics for depression, self-evaluated Beck Depression Inventory (BDI) scores, and previous history and family history of depression were obtained. Cardiovascular characteristics were evaluated for ACS diagnosis (MI or unstable angina), previous history and family history of ACS, hypertension and diabetes diagnosis, and reported current smoking status. The following cardiovascular risk markers were also evaluated: echocardiography for evaluating left ventricular ejection fraction and wall motion scores, electrocardiography for evaluating heart rate, PR interval, QRS duration and QTc duration, body mass index (BMI), and blood pressure. Also, laboratory tests for troponin I, creatine kinase-MB, tumor necrosis factor alpha (TNF-α), interleukin (IL)-1β, IL-6, IL-18, highly sensitive C-reactive protein (hs-CRP), homocysteine, total cholesterol, low-density lipoprotein (LDL) and triglyceride were investigated.

3. Long-Term Prognosis

The long-term cardiac prognosis was comprehensively evaluated using KAMIR data containing accumulated detailed electronic information on hospitalization, death, recurrent myocardial infarction, and percutaneous coronary intervention. All participants were followed up for such prognosis in the present study. In order to enable non-hierarchic endpoint analysis among survival analyses, all patients were followed up until the time point of interest or death. The primary endpoint was a major adverse cardiac event (MACE), which included all-cause mortality, myocardial infarction (MI) and percutaneous coronary intervention (PCI). Secondary endpoints were all-cause mortality, cardiac death (defined as sudden death without other causes, death from arrhythmia, myocardial infarction (MI) or heart failure, or death caused by heart surgery or endocarditis), myocardial infarction (MI) and percutaneous coronary intervention (PCI). All potential events were finalized by an independent endpoint committee consisting of study cardiologists who were blind to participants' depression comorbidity.

4. Statistical Analysis

Using the median values of the participants, the NR3C1 methylation percentages were classified by a binary variable (lower and higher categories). The demographic and clinical characteristics of ACS patients having lower and higher methylation levels were compared applying t-test or χ2 test, as appropriate. The association between methylation percentages and cardiovascular risk markers was calculated using the Spearman correlation coefficient after partial for the BDI score. Factors potentially associated with the extent of methylation (P<0.05) and other variables known to have a potential effect on MACE were used as covariates in subsequent analysis. The Kaplan-Meier curve was calculated, and the cumulative distribution of MACE in ACS patients having lower and higher methylation levels was compared through a log-rank method and further stratified by the depression status at baseline. The time to the first composite and individual MACEs was compared after correction of potential covariates between the two groups described above using the Cox proportional hazards model. In order to reexamine the effect thereof beyond the binary categorical approach, additional sensitivity analysis was performed using methylation values as continuous exposure variables (in increments of 10%) and as tertials (lower, middle, and higher groups). A Schoenfeld residual test was used to verify the proportional hazards assumption in all models. All statistical tests were two-sided with a significance level of 0.05. Statistical analysis was performed using SPSS 21.0 and STATA 12.0 software.

5. Results
(1) Extent of NR3C1 Methylation and Baseline Characteristics

Among 969 participants, 378 (39%) ACS patients experienced depression at baseline. The median (interquartile range) and mean (standard deviation) values of individual methylations and average methylation of the CpG1, CpG2, and CpG3 sites are summarized in Table 1 below.

TABLE 1

| | Median (interquartile range) | Mean (standard deviation) |
|---|---|---|
| CpG average | 20.4 (12.4-28.4) | 21.7 (12.0) |
| CpG1 | 15.9 (9.9-23.7) | 17.3 (9.9) |
| CpG2 | 24.3 (14.0-34.0) | 25.2 (15.1) |
| CpG3 | 23.0 (11.0-30.0) | 22.7 (13.5) |

As is apparent from Table 1, since the NR3C1 methylation percentages of the three sites are closely related to each other (all Spearman's rho>0.7, p-value<0.001) and also, since the average value is more accurate than the individual values and similar to the individual values, the average value of the three sites was used as a single value in subsequent analysis.

In addition, baseline characteristics of ACS patients having lower or higher NR3C1 methylation levels were compared in Table 2 below.

TABLE 2

| | Lower | | Higher | | | |
|---|---|---|---|---|---|---|
| | methylation | | methylation | | Statistical | |
| | (N = 484) | | (N = 485) | | coefficient | P-value^{a} |
| Socio-demographic characteristics | | | | | | |
| Age, mean (SD) years | 58.7 | (11.2) | 57.8 | (11.0) | t = +1.012 | 0.312 |
| Gender, N (%) female | 349 | (72.1) | 351 | (72.4) | χ² = 0.008 | 0.927 |
| Education, mean (SD) years | 9.9 | (4.7) | 9.8 | (4.6) | t = +0.447 | 0.655 |
| Unmarried marital status, N (%) | 68 | (14.0) | 73 | (15.1) | χ² = 0.196 | 0.658 |
| Living alone, N (%) | 41 | (8.5) | 51 | (10.5) | χ² = 1.178 | 0.278 |
| Housing, N (%) rented | 68 | (14.0) | 82 | (16.9) | χ² = 1.512 | 0.210 |
| Currently unemployed, N (%) | 179 | (37.0) | 189 | (39.0) | χ² = 0.405 | 0.524 |
| Depression characteristics | | | | | | |
| BDI, mean (SD) score | 8.3 | (8.0) | 11.8 | (8.9) | t = −6.483 | <0.001 |
| Previous depression, N (%) | 15 | (3.1) | 19 | (3.9) | χ² = 0.479 | 0.489 |
| Family history of depression, N (%) | 15 | (3.1) | 8 | (1.6) | χ² = 2.197 | 0.138 |
| Cardiac characteristics, N (%) | | | | | | |
| ACS diagnosis | | | | | | |
| Myocardial infarction | 368 | (76.0) | 346 | (71.3) | χ² = 2.751 | 0.097 |
| Unstable angina | 116 | (24.0) | 139 | (28.7) | | |
| Previous ACS | 17 | (3.5) | 22 | (4.5) | χ² = 0.657 | 0.418 |
| Family history of ACS | 14 | (2.9) | 17 | (3.5) | χ² = 0.294 | 0.588 |
| Hypertension | 224 | (46.3) | 234 | (48.2) | χ² = 0.376 | 0.540 |
| Diabetes mellitus | 90 | (18.6 | 101 | (20.8) | χ² = 0.761 | 0.383 |
| Current smoker | 174 | (36.0) | 192 | (39.6) | χ² = 1.363 | 0.243 |

^{a}p-values using t-tests or χ² tests as appropriate.
BDI, Beck Depression Inventory; ACS, acute coronary syndrome.

The mean (standard deviation) and range of the group having lower methylation, were 12.2 (5.2) and 0-20.4%, respectively, while those of the group having higher methylation were 31.2 (9.1) and 20.4-66.0%, respectively. As is apparent from Table 2, higher methylation was only significantly associated with the high BDI score. There was no difference in clinical characteristics between the participants who agreed to provide blood samples and those who did not, in view of the baseline characteristics (all p-values>0.15).

The reference level was determined to be 21%, in consideration of the median and mean values of the average methylation percentage of three CPG sites of the NR3C1 gene for 969 patients at baseline as shown in Table 1, and the results of Table 2.

(2) Association of NR3C1 Methylation and Cardiovascular Risk Marker

The average NR3C1 methylation value was associated with longer QTc duration, higher BMI, and higher troponin I and CK-MB concentrations even after partial for the BDI score (Table 3). These characteristics were used as covariates in subsequent analysis. Moreover, age, ACS diagnosis, previous ACS, hypertension, diabetes, smoking, LVEF, and depressive disorder were also used in analysis as covariates in consideration of association with cardiac prognosis reported in previous studies.

TABLE 3

| | rho | P-value |
|---|---|---|
| Left ventricular ejection fraction, mean (SD) % | −0.040 | 0.210 |
| Wall motion, mean(SD) score | +0.051 | 0.110 |
| Heart rate, mean (SD) beats/min | +0.057 | 0.079 |
| PR interval, mean (SD) ms | +0.054 | 0.090 |
| QRS duration, mean (SD) ms | +0.004 | 0.909 |
| QTc duration, mean (SD) ms | +0.069 | 0.036 |
| Body mass index, mean (SD) Kg/m² | +0.073 | 0.025 |
| Systolic blood pressure, mean (SD) mmHg | −0.001 | 0.987 |
| Diastolic blood pressure, mean (SD) mmHg | +0.015 | 0.650 |
| Troponin I, mean(SD) mg/dL | +0.066 | 0.041 |
| Creatine kinase-MB, mean(SD) mg/dL | +0.066 | 0.040 |
| Tumor necrosis factor-a, mean(SD) pg/mL | +0.040 | 0.211 |
| Interleukin-1b, mean(SD) pg/mL | +0.009 | 0.779 |
| Interleukin-6, mean(SD) pg/mL | −.0.009 | 0.770 |
| Interleukin-18, mean(SD) pg/mL | +0.018 | 0.574 |
| High sensitivity C-reactive protein, mean(SD) | +0.050 | 0.123 |
| mg/dL | | |
| Homocysteine, mean(SD) μmol/L | −0.002 | 0.959 |
| Total cholesterol, mean(SD) mg/dL | +0.021 | 0.513 |
| Lower density lipoprotein cholesterol, mean(SD) | +0.010 | 0.774 |
| mg/dL | | |
| Triglyceride, mean(SD) mg/dL | −0.007 | 0.845 |

(3) Effect of NR3C1 Methylation on ACS Prognosis

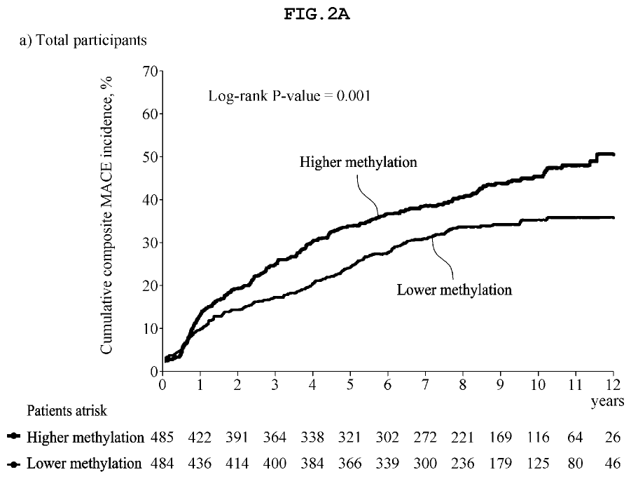

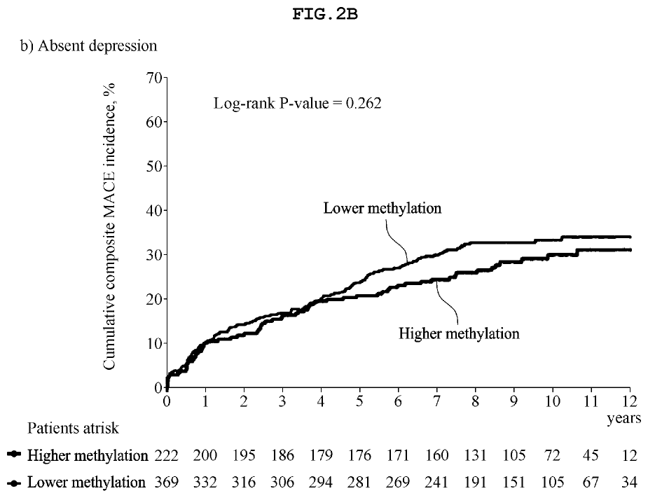

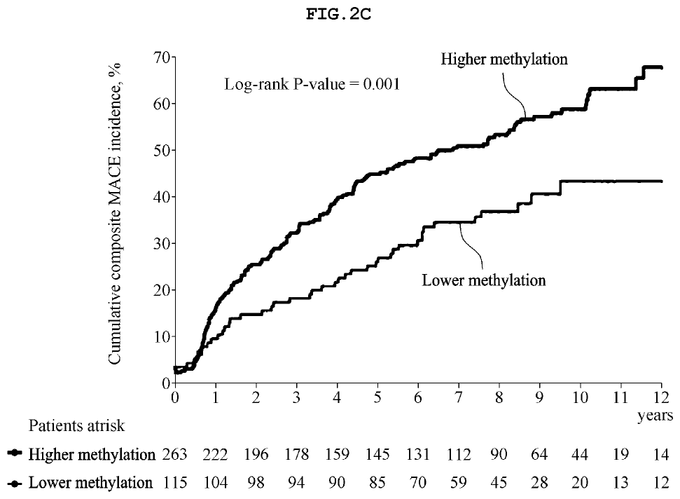

All participants were followed up for cardiac prognosis until 2017 or until to death [follow-up period median; mean (standard deviation)=8.4; 8.7 (1.5) years]. 383 (39.5%) participants experienced composite MACE, which is the primary endpoint. The number of participants experiencing secondary endpoints was as follows: all-cause mortality was reported in 178 participants (18.4%), cardiac death was reported in 98 participants (10.1%), myocardial infarction was reported in 101 participants (10.4%), and PCI was reported in 139 participants (14.3%). The cumulative risk for composite MACE in ACS patients having lower and higher average NR3C1 methylation levels is depicted in FIGS. 2A to 2C. Significant differences were found in all participants, and when stratified for depression status, a significant difference was found only in the group with depression, and no difference was found in the group without depression.

The comparison of the first MACE rates by lower and higher NR3C1 methylation levels after successively adjusted for potential covariates is shown in Table 4 below.

TABLE 4

| | N (%) MACE | | | | |
|---|---|---|---|---|---|
| | Lower | Higher | Hazard ratios (95% confidence intervals) | | |
| | methylation | methylation | [HRs (95% CIs)] | | |
| | (N = 484) | (N = 485) | Unadusted | Adjusted^{a} | Adjusted^{b} |
| MACE | 166 (34.3) | 217 (44.7) | 1.48 (1.20-1.81)^{‡} | 1.40 (1.14-1.71) ^{†} | 1.25 (1.01-1.55)* |
| All-cause | 76 (15.7) | 102 (21.0) | 1.40 (1.04-1.89)* | 1.35 (0.99-1.81) | 1.24 (0.90-1.70) |
| mortality | | | | | |
| Cardiac death | 42 (8.7) | 56 (11.5) | 1.40 (0.94-2.11) | 1.34 (0.90-1.99) | 1.23 (0.80-1.88) |
| Myocardial | 41 (8.5) | 60 (12.4) | 1.51 (1.01-2.26)* | 1.49 (1.99-2.19)* | 1.17 (0.77-1.78) |
| infarction | | | | | |
| Percutaneous | 60 (12.4) | 79 (16.3) | 1.45 (1.03-2.04)* | 1.38 (0.99-1.93) | 1.24 (0.87-1.77) |
| coronary | | | | | |
| intervention | | | | | |

^{a}Model 1: adjusted for age, ACS diagnosis, previous ACS, hypertension, diabetes, smoking, left ventricular ejection fraction, QTc duration, body mass index, and serum levels of troponin I and creatine kinase-MB.
^{b}Model 2: additionally adjusted for depressive disorder status.
*p-value < 0.05;
^{†} p-value < 0.01;
^{‡}p-value < 0.001.

As is apparent from Table 4, higher methylation was associated with higher rates of composite MACE, all-cause mortality, MI, and PCI in unadjusted analysis. Moreover, after adjustment of age, ACS diagnosis, previous ACS, hypertension, diabetes, smoking, LVEF, QTc duration, BMI, serum levels of troponin I and blood levels of creatine kinase-MB, the association thereof became weak, and thus only the association with composite MACE and MI remained significant. After further adjustment of depression status, the association became weaker, and a significant association was observed only with the prognosis of composite MACE.

The comparison of the first MACE rates by lower and higher NR3C1 methylation levels was stratified for baseline depression status and adjusted for potential covariates, and the results thereof are shown in Table 5 below.

TABLE 5

| | Absent depressive disorder (N = 591) | | | Present depressive disorder (N = 378) | | | |
|---|---|---|---|---|---|---|---|
| | N (%) MACE | | | N (%) MACE | | | |
| | Lower | Higher | | Lower | Higher | | |
| | methylation | methylation | HRs | methylation | methylation | HRs | P-value for |
| | (N = 369) | (N = 222) | (95% CIs) | (N = 115) | (N = 263) | (95% CIs) | interaction |
| Composite | 122 (33.1) | 64 (28.8) | 0.97 (0.83-1.13) | 44 (38.3) | 153 (58.2) | 1.98 (1.39-2.81)^{‡} | 0.001 |
| MACE | | | | | | | |
| All-cause | 58 (15.7) | 29 (13.1) | 0.95 (0.76-1.20) | 18 (15.7) | 73 (27.8) | 1.90 (1.12-3.22)* | 0.013 |
| mortality | | | | | | | |
| Cardiac | 32 (8.7) | 14 (6.3) | 0.89 (0.64-1.22) | 10 (8.7) | 42 (16.0) | 2.24 (1.09-4.59)* | 0.036 |
| death | | | | | | | |
| Myocardial | 29 (7.9) | 16 (7.2) | 0.93 (0.69-1.27) | 12 (10.4) | 44 (16.7) | 1.78 (0.92-3.46) | 0.177 |
| infarction | | | | | | | |
| Percutaneous | 43 (11.7) | 23 (10.4) | 0.95 (0.73-1.22) | 17 (14.8) | 56 (21.3) | 1.90 (1.09-3.32)* | 0.157 |
| coronary | | | | | | | |
| intervention | | | | | | | |

Hazard ratio (95% confidence interval) [HR (95% CI)] was estimated adjusted for age, ACS diagnosis, previous ACS, hypertension, diabetes, smoking, left ventricular ejection fraction, QTc duration, body mass index, and serum levels of troponin I and creatine kinase-MB at baseline.
*p-value < 0.05;
^{†}p-value < 0.01;
^{‡}p-value < 0.001.

As is apparent from Table 5, higher methylation was significantly associated with higher rates of composite MACE, all-cause mortality, cardiac death, and PCI only in those with depressive disorder after adjustment for age, ACS diagnosis, previous ACS, hypertension, diabetes, smoking, LVEF, QTc duration, BMI, and serum levels of troponin I and creatine kinase-MB. In view of the multiplicative interaction between NR3C1 methylation and depressive disorder, it was significantly associated only with composite MACE, all-cause mortality, and cardiac death.

The results of additional sensitivity analysis assessed using the extent of methylation as a continuous variable or as tertials (lower, middle, and higher) are shown in Tables 6 and 7 below.

TABLE 6

| | Absent | Present |
|---|---|---|
| | depressive disorder | depressive disorder |
| | (N = 591) | (N = 378) |
| Major adverse cardiac | 0.97 (0.92-1.02) | 1.11 (1.08-1.17)‡ |
| events | | |
| All-cause mortality | 0.98 (0.91-1.06) | 1.08 (1.02-1.16)* |
| Cardiac death | 0.98 (0.88-1.01) | 1.11 (1.02-1.20)† |
| Myocardial infarction | 0.94 (0.84-1.05) | 1.07 (1.00-1.14)* |
| Percutaneous coronary | 0.96 (0.88-1.05) | 1.12 (1.02-1.22)* |
| intervention | | |

Data were adjusted at baseline for age, Beck Depression Inventory Score, ACS diagnosis, previous ACS, hypertension, diabetes, smoking, QTc duration, left ventricular excretion fraction, body mass index, and serum levels of troponin I and creatine kinase-MB.
*p-value < 0.05;
†p-value < 0.01;
‡p-value < 0.001.

TABLE 7

| | Average | Absent | Present |
|---|---|---|---|
| | NR3C1 | depressive disorder | depressive disorder |
| Outcomes | methylation | (N = 591) | (N = 378) |
| Major adverse | Lower | Ref | Ref |
| cardiac events | Middle | 1.00 (0.71-1.39) | 0.88 (0.55-1.41) |
| | Higher | 0.73 (0.48-1.11) | 1.48 (1.02-2.13)* |
| All-cause | Lower | Ref | Ref |
| mortality | Middle | 0.97 (0.59-1.60) | 1.11 (0.55-2.26) |
| | Higher | 0.86 (0.47-1.55) | 1.58 (0.91-2.76) |
| Cardiac death | Lower | Ref | Ref |
| | Middle | 1.45 (0.73-2.88) | 1.24 (0.46-3.35) |
| | Higher | 0.74 (0.30-1.85) | 1.75 (0.81-3.78) |
| Myocardial | Lower | Ref | Ref |
| infarction | Middle | 1.04 (0.54-1.97) | 1.39 (0.55-3.51) |
| | Higher | 0.50 (0.20-1.25) | 1.78 (0.84-3.79) |
| Percutaneous | Lower | Ref | Ref |
| coronary | Middle | 0.90 (0.52-1.58) | 0.71 (0.33-1.53) |
| intervention | Higher | 0.61 (0.30-1.27) | 1.53 (0.84-2.78) |

Data were adjusted at baseline for age, Beck Depression Inventory Score, ACS diagnosis, previous ACS, hypertension, diabetes, smoking, QTc duration, left ventricular excretion fraction, body mass index, and serum levels of troponin I and creatine kinase-MB.
*p-value < 0.05;
†p-value < 0.01;
‡p-value < 0.001.

Summarizing the results of Tables 6 and 7, the strength of the association were not changed using the continuous variable, but in the analysis using methylation status as tertials, only the association with the composite MACE after covariate adjustment was significant in patients with depressive disorder, and the remaining associations were not significant. Model assumption was appropriate in all Cox hazards analyses (Schoenfeld p values>030).
(4) Conclusion

The above-described results show that higher methylation status than the reference level measured at baseline is associated with several cardiovascular risk markers in the acute phase of ACS and predicted poor long-term prognosis. This longitudinal association was significant only in the presence of depression at baseline, with significant synergistic interaction, and this association was independent of a range of potential covariates. Moreover, NR3C1 hypermethylation in ACS patients was significantly associated with cardiovascular risk markers such as QTc duration, BMI, and serum troponin I and CK-MB. These results are consistent with recent results reporting an association of NR3C1 hypermethylation with atherosclerosis and with heightened cardiovascular reactivity.

An association of NR3C1 hypermethylation equal to or greater than the reference level with ACS long-term prognosis was more strongly explained by depression status. A significant association between methylation and long-term prognosis was independently associated with cardiovascular risk factors only in the presence of depression. This synergistic effect may be explained by the following mechanisms. From a biological aspect, NR3C1 hypermethylation is associated with the adverse profiles of cardiovascular risk markers, and depression itself is associated with changes in pro-inflammatory cytokines and with dysfunction of the autonomic nervous system and platelets, and these changes may have an adverse impact on cardiac prognosis. This synergic effect probably reflects multiple risk pathway involvement. In consideration of a behavioral aspect, NR3C1 hypermethylation has a functional effect on GR expression and ultimately causes dysregulation in the stress response through HPA axis, which may reduce the ability to cope with various stressful situations encountered by ACS patients suffering from unexpected ACS, financial and occupational problems, and difficulties in daily living, as well as other ACS patients. Depression is also associated with unhealthy lifestyles, such as a sedentary lifestyle, irregular hospital visits and medication, which may have negative affect on cardiac prognosis.

As a result, NR3C1 hypermethylation at baseline in ACS patients can predict poor long-term prognosis in the presence of depression, independent of potential covariates, including cardiovascular risk markers. Similarly, the association between depression and poor long-term prognosis is stronger in ACS patients having NR3C1 hypermethylation. The NR3C1 methylation test at baseline has clinical significance in screening epigenetic risk to distinguish the high-risk group with poor ACS prognosis.

Therefore, in the present disclosure, the extent of NR3C1 methylation can be provided as a promising biomarker for predicting long-term prognosis of acute coronary syndrome when ACS patients are diagnosed with depression at baseline.

Specifically, by measuring the extent of NR3C1 methylation in ACS patients with depression at baseline, clinical prediction of long-term cardiac prognosis 5 years after baseline can be improved.

As described above, the use of NR3C1 methylation at baseline as a biomarker for predicting long-term prognosis of acute coronary syndrome in view of clinical implications provides advantages of non-invasiveness and convenience, and thus the present disclosure is capable of more easily confirming the possibility of incidence of major adverse cardiac events, including recurrence and/or death, after acute coronary syndrome.

Consequently, the present disclosure can not only contribute to a decision-making process pertaining to therapeutic drugs or treatment methods, but also can be very helpful as a potential tool for preemptively preventing recurrence and/or death after acute coronary syndrome in a patient.

Although preferred embodiments of the present disclosure have been disclosed for illustrative purposes, those skilled in the art will appreciate that various modifications and substitutions are possible without departing from the scope and spirit of the disclosure as disclosed in the accompanying claims.

A sequence listing electronically submitted with the present application on Feb. 5, 2021 as an ASCII text file named 20210205_Q48021IM01_TU_SEQ, created on Feb. 5, 2021 and having a size of 1,000 bytes, is incorporated herein by reference in its entirety.

What is claimed is:

1. A method of determining long-term prognosis of acute coronary syndrome associated with depression, comprising:
an investigation step of confirming whether a patient with acute coronary syndrome has depression at a baseline;
a measurement step of measuring a level or amount of a biomarker for predicting long-term prognosis of acute coronary syndrome associated with depression contained in a biological sample of the patient confirmed to have depression in the investigation step; and
a decision step of determining a risk of onset of a major adverse cardiac event including recurrence or death after acute coronary syndrome based on the level or amount of the biomarker for predicting long-term prognosis of acute coronary syndrome associated with depression measured in the measurement step.

2. The method of claim 1, wherein the biomarker for predicting long-term prognosis of acute coronary syndrome associated with depression is methylation of a CpG region contained in a nucleotide region at 5′-end positions from −3166 to −3147 of an NR3C1 gene.

3. The method of claim 2, wherein the decision step is performed by comparing an extent of methylation of the CpG region contained in the nucleotide region at 5′-end positions from −3166 to −3147 of the NR3C1 gene measured in the measurement step with a preset reference level, and the reference level is determined depending on an extent of methylation of three CpG sites contained in a nucleotide region at 5′-end positions from −3166 to −3147 of an NR3C1 gene obtained from a patient population with acute coronary syndrome at the baseline.

4. The method of claim 3, wherein the three CpG sites are CpG1, CpG2 and CpG3, and the reference level is 21%, determined in consideration of median and mean values of an average methylation percentage of CpG1, CpG2 and CpG3.

5. The method of claim 4, wherein, in the decision step, it is determined that there is a risk of onset of the major adverse cardiac event 5 years after the baseline when an average methylation value of CpG1, CpG2 and CpG3 measured is equal to or greater than the reference level.

6. The method of claim 2, wherein, when the extent of methylation of the CpG region is increased, the risk of onset of the major adverse cardiac event is increased.

7. The method of claim 3, wherein, when the extent of methylation of the CpG region is increased, the risk of onset of the major adverse cardiac event is increased.

8. The method of claim 4, wherein, when the extent of methylation of the CpG region is increased, the risk of onset of the major adverse cardiac event is increased.

9. The method of claim 5, wherein, when the extent of methylation of the CpG region is increased, the risk of onset of the major adverse cardiac event is increased.

10. The method of claim 6, wherein, when an average methylation value of CpG1, CpG2, and CpG3 in the CpG region is increased by 10% over the reference level, the risk of onset of the major adverse cardiac event is increased by 11%.

11. The method of claim 7, wherein, when an average methylation value of CpG1, CpG2, and CpG3 in the CpG region is increased by 10% over the reference level, the risk of onset of the major adverse cardiac event is increased by 11%.

12. The method of claim 8, wherein, when an average methylation value of CpG1, CpG2, and CpG3 in the CpG region is increased by 10% over the reference level, the risk of onset of the major adverse cardiac event is increased by 11%.

13. The method of claim 9, wherein, when an average methylation value of CpG1, CpG2, and CpG3 in the CpG region is increased by 10% over the reference level, the risk of onset of the major adverse cardiac event is increased by 11%.

14. The method of claim 1, wherein the biological sample is selected from among a tissue and a body fluid including blood.

15. A diagnostic kit for determining long-term prognosis of acute coronary syndrome, comprising:
a measurement means for measuring a biomarker for predicting long-term prognosis of acute coronary syndrome associated with depression, which measures methylation of a CpG region contained in a nucleotide region at 5′-end positions from −3166 to −3147 of an NR3C1 gene of a patient with acute coronary syndrome confirmed to have depression at a baseline.

16. The diagnostic kit of claim 15, wherein the measurement means uses sodium bisulfate and a polymerase chain reaction (PCR) or uses a monoclonal antibody against 5-methylcytosine.

17. The diagnostic kit of claim 15, wherein the CpG region comprises CpG1, CpG2 and CpG3.

18. The diagnostic kit of claim 15, wherein the diagnostic kit is a microarray.

19. The diagnostic kit of claim 17, wherein the diagnostic kit is a microarray.


SEQUENCE LISTING (1 sequence)

SEQ ID NO: 1 - DNA, 412 bp, Homo sapiens
tcccttccctgaagcctccccagagggcgtgtcaggccgcccggccccgagcgcggccgagacgctgcggcaccgtttccgtgcaaccccgtagcccctttcgaagtgacacacttcacgcaactcggcccggcggcggcggcgcgggccactcacgcagctcagccgcgggaggcgccccggctcttgtggcccgcccgctgtcacccgcaggggcactggcggcgcttgccgccaaggggcagagcgagctcccgagtgggtctggagccgcggagctgggcgggggcgggaaggaggtagcgagaaaagaaactggagaaactcggtggccctcttaacgccgccccagagagaccaggtcggcccccgccgctgccgccgccaccctttttcctggggagttgggggc